(19) United States
(12) Patent Application Publication
(10) Pub. No.: US 2009/0028791 A1
(43) Pub. Date: Jan. 29, 2009

(54) Dichloroacetate Analogs as Imaging Agents

(76) Inventors: Julius A. Balatoni, Houston, TX (US); Louis R. DePalatis, Lake Jackson, TX (US); William Tong, Houston, TX (US); Juri Gelovani, Pearland, TX (US)

Correspondence Address: WILLIAMS, MORGAN & AMERSON, 10333 RICHMOND, SUITE 1100, HOUSTON, TX, 77042, US

(21) Appl. No.: 12/178,826

(22) Filed: Jul. 24, 2008

Related U.S. Application Data
(60) Provisional application No. 60/951,773, filed on Jul. 25, 2007.

(51) Int. Cl. A61K 51/04; C07C 69/63; A61K 31/22; A61P 35/00

(52) U.S. Cl. 424/1.89; 560/226; 424/1.85; 514/550

(57) ABSTRACT

A compound having structure I:

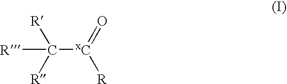

wherein ^{x}C is selected from the group consisting of ¹¹C, ¹²C, and ¹³C; R is selected from the group consisting of —OH, —O⁻R⁴⁺ wherein R⁴ is selected from the group consisting of Na and K, —OR³ wherein R³ is selected from the group consisting of C₁₋₈ alkyl and C₁₋₈ alkenyl, —NH₂, and NHR³; R′ is selected from the group consisting of —H, —Cl, —F, —Br, and —I; R″ is selected from the group consisting of —H, —Cl, —F, —Br, and —I; and R′″ is selected from the group consisting of —H, —Cl, —F, —Br, —I, ¹⁸F, ¹⁹F, ⁷⁵Br, ⁷⁶Br, ⁷⁷Br, ¹²³I, ¹²⁴I, and ¹³¹I; provided, when ^{x}C is ¹²C, R′″ is selected from the group consisting of ¹⁸F, ¹⁹F, ⁷⁵Br, ⁷⁶Br, ⁷⁷Br, ¹²³I, ¹²⁴I, and ¹³¹I. Also, a method of imaging in a mammal a medical condition, such as a cancer, lactic acidosis, or cardiac or cardiovascular function by administering to the mammal a composition comprising a compound having structure I and observing the distribution of the compound in the mammal by an imaging modality selected from the group consisting of positron emission tomography (PET) imaging, single photon emission computed tomography (SPECT), gamma camera devices, magnetic resonance imaging (MRI), and hand-held radiation detection probes. In addition, a method of treating a cancer in a mammal by administering to the mammal a composition comprising a compound having structure I.

This application claims priority from United States provisional patent application Ser. No. 60/951,773, filed on Jul. 25, 2007, which is incorporated herein by reference.

### BACKGROUND OF THE INVENTION

The present invention relates generally to the fields of cancer diagnosis and treatment. More particularly, it concerns the use of an imaging modality selected from the group consisting of positron emission tomography (PET) imaging, single photon emission computed tomography (SPECT), gamma camera devices, magnetic resonance imaging (MRI), and hand-held radiation detection probes in the diagnosis of cancer.

In PET, an isotope which decays by emitting a positron is chemically incorporated into a metabolically active molecule and injected into a living subject. Injection can be performed into any fluid or tissue, such as blood or a tumor, among others. After injection, the metabolically active molecule becomes concentrated in tissues of interest that contain molecules, enzymes, or other structures which interact with the metabolically active molecule and the subject is placed in proximity to the imaging device. Decay of the short-lived isotope emits a positron. After traveling a short distance (typically no more than a few millimeters) the positron collides with an electron and undergoes an annihilation event, producing a pair of gamma photons moving in opposite directions. These are detected when they reach a scintillator material in the scanning device, creating a burst of light which is detected by photomultiplier tubes.

The most significant fraction of electron-positron decays result in two 511 keV photons being emitted at almost 180 degrees to each other, allowing localization of their source along a straight line of coincidence. Using statistics collected from tens-of-thousands of coincidence events, a set of simultaneous equations for the activity of each parcel of tissue along many lines of coincidence can be solved, and thus a map of locations and radioactivities in the body may be plotted. The resulting map shows the tissues in which the molecular probe has become concentrated, and can be interpreted by nuclear medicine physician or radiologist in the context of the patient's diagnosis or treatment plan.

Other imaging techniques, including but not limited to single photon emission computed tomography (SPECT), gamma camera devices, magnetic resonance imaging (MRI), and hand-held radiation detection probes, are also known.

Because such isotopes typically have half-lives of tens of minutes or less, compounds incorporating such isotopes must generally be synthesized shortly before use, in contrast to compounds incorporating longer-lived isotopes.

### SUMMARY OF THE INVENTION

In one embodiment, the present invention relates to a compound having structure I:

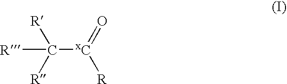

wherein ^{x}C is selected from the group consisting of ¹¹C, ¹²C, and ¹³C; R is selected from the group consisting of —OH, —O⁻R⁴⁺ wherein R⁴ is selected from the group consisting of Na and K, —OR³ wherein R³ is selected from the group consisting of C₁₋₈ alkyl and C₁₋₈ alkenyl, —NH₂, and NHR³; R′ is selected from the group consisting of —H, —Cl, —F, —Br, and —I; R″ is selected from the group consisting of —H, —Cl, —F, —Br, and —I; and R′″ is selected from the group consisting of —H, —Cl, —F, —Br, —I, ¹⁸F, ¹⁹F, ⁷⁵Br, ⁷⁶Br, ⁷⁷Br, ¹²³I, ¹²⁴I, and ¹³¹I; provided, when ^{x}C is ¹²C, R′″ is selected from the group consisting of ¹⁸F, ¹⁹F, ⁷⁵Br, ⁷⁶Br, ⁷⁷Br, ¹²³I, ¹²⁴I, and ¹³¹I.

In one embodiment, the present invention relates to a method of imaging in a mammal a cancer by administering to the mammal a composition comprising a compound having structure I, as described above, and observing the distribution of the compound in the mammal by an imaging modality selected from the group consisting of positron emission tomography (PET) imaging, single photon emission computed tomography (SPECT), gamma camera devices, magnetic resonance imaging (MRI), and hand-held radiation detection probes.

In another embodiment, the present invention relates to a method of treating a cancer in a mammal by administering to the mammal a composition comprising a compound having structure I, as described above.

Dichloroacetate analogs, such as those described herein, may be useful to image all tumor types, including primary and metastatic tumors.

### DESCRIPTION OF ILLUSTRATIVE EMBODIMENTS

In one embodiment, the present invention relates to a compound having structure I:

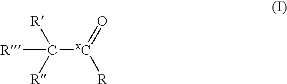

wherein ^{x}C is selected from the group consisting of ¹¹C, ¹²C, and ¹³C; R is selected from the group consisting of —OH, —O⁻R⁴⁺ wherein R⁴ is selected from the group consisting of Na and K, —OR³ wherein R³ is selected from the group consisting of C₁₋₈ alkyl and C₁₋₈ alkenyl, —NH₂, and NHR³; R′ is selected from the group consisting of —H, —Cl, —F, —Br, and —I; R″ is selected from the group consisting of —H, —Cl, —F, —Br, and —I; and R′″ is selected from the group consisting of —H, —Cl, —F, —Br, —I, ¹⁸F, ¹⁹F, ⁷⁵Br, ⁷⁶Br, ⁷⁷Br, ¹²³I, ¹²⁴I, and ¹³¹I; provided, when ^{x}C is ¹²C, R′″ is selected from the group consisting of ¹⁸F, ¹⁹F, ⁷⁵Br, ⁷⁶Br, ⁷⁷Br, ¹²³I, ¹²⁴I, and ¹³¹I.

Herein, element symbols shown in the absence of an atomic weight superscript (e.g., C, Cl, F, Br, and I) represent any isotope of the element. Nuclide symbols shown with an atomic weight superscript (e.g., ¹¹C, ¹⁸F, ¹⁹F, ⁷⁵Br, ⁷⁶Br, ⁷⁷Br, ¹²³I, ¹²⁴I, and ¹³¹I) represent the particular isotope having that atomic weight.

Any physiologically tolerable ion can be used as the counterion to —O⁻ in the R group. In one embodiment, the physiologically tolerable ion is selected from the group consisting of Na⁺ and K⁺. Na⁺ and K⁺ have been used in dichloroacetate salts for treatment of lactic acidosis and inhibition of cancer cells in vitro.

In a further embodiment, the compound can be selected from the group consisting of (i) the compound having structure I, wherein ^{x}C is ¹¹C, R is —O⁻R⁴⁺ wherein R⁴ is selected from the group consisting of Na and K, R′ is —Cl, R″ is —H, and R′″ is —Cl; (ii) the compound having structure I, wherein ^{x}C is ¹²C, R is —O⁻R⁴⁺ wherein R⁴ is selected from the group consisting of Na and K, R′ is —Cl, R″ is —H, and R′″ is ¹⁸F; (iii) the compound having structure I, wherein ^{x}C is ¹²C, R is —O⁻R⁴⁺ wherein R⁴ is selected from the group consisting of Na and K, R′ is —Cl, R″ is —Cl, and R′″ is ¹⁸F; (iv) the compound having structure I, wherein ^{x}C is ¹²C, R is —O⁻R⁴⁺ wherein R⁴ is selected from the group consisting of Na and K, R′ is —Cl, R″ is —H, and R′″ is ⁷⁵Br; (v) the compound having structure I, wherein ^{x}C is ¹²C, R is —O⁻R⁴⁺ wherein R⁴ is selected from the group consisting of Na and K, R′ is —Cl, R″ is —H, and R′″ is ⁷⁶Br; (vi) the compound having structure I, wherein ^{x}C is ¹²C, R is —O⁻R⁴⁺ wherein R⁴ is selected from the group consisting of Na and K, R′ is —Cl, R″ is —H, and R′″ is ⁷⁷Br; (vii) the compound having structure I, wherein ^{x}C is ¹²C, R is —O⁻R⁴⁺ wherein R⁴ is selected from the group consisting of Na and K, R′ is —Cl, R″ is —Cl, and R′″ is ⁷⁵Br; (viii) the compound having structure I, wherein ^{x}C is ¹²C, R is —O⁻R⁴⁺ wherein R⁴ is selected from the group consisting of Na and K, R′ is —Cl, R″ is —Cl, and R′″ is ⁷⁶Br; (ix) the compound having structure I, wherein ^{x}C is ¹²C, R is —O⁻R⁴⁺ wherein R⁴ is selected from the group consisting of Na and K, R′ is —Cl, R″ is —Cl, and R′″ is ⁷⁷Br; (x) the compound having structure I, wherein ^{x}C is ¹²C, R is —O⁻R⁴⁺ wherein R⁴ is selected from the group consisting of Na and K, R′ is —Cl, R″ is —H, and R′″ is ¹²³I; (xi) the compound having structure I, wherein ^{x}C is ¹²C, R is —O⁻R⁴⁺ wherein R⁴ is selected from the group consisting of Na and K, R′ is —Cl, R″ is —H, and R′″ is ¹²⁴I; (xii) the compound having structure I, wherein ^{x}C is ¹²C, R is —O⁻R⁴⁺ wherein R⁴ is selected from the group consisting of Na and K, R′ is —Cl, R″ is —H, and R′″ is ¹³¹I; (xiii) the compound having structure I, wherein ^{x}C is ¹²C, R is —O⁻R⁴⁺ wherein R⁴ is selected from the group consisting of Na and K, R′ is —Cl, R″ is —Cl, and R′″ is ¹²³I; (xiv) the compound having structure I, wherein ^{x}C is ¹²C, R is —O⁻R⁴⁺ wherein R⁴ is selected from the group consisting of Na and K, R′ is —Cl, R″ is —Cl, and R′″ is ¹²⁴I; and (xv) the compound having structure I, wherein ^{x}C is ¹²C, R is —O⁻R⁴⁺ wherein R⁴ is selected from the group consisting of Na and K, R′ is —Cl, R″ is —Cl, and R′″ is ¹³¹I.

Of the specific isotopes referred to above, ¹¹C has a half life of about 20 min, ¹⁸F has a half life of about 110 min, ⁷⁵Br has a half life of about 95 min ⁷⁶Br has a half life of about 1000 min ⁷⁷Br has a half life of about 3400 min, ¹²³I has a half life of about 780 min, ¹²⁴I has a half life of about 6000 min, and ¹³¹I has a half life of about 11,500 min.

In one embodiment, the present invention relates to a method of imaging in a mammal a cancer by administering to the mammal a composition comprising a compound having structure I, as described above, and observing the distribution of the compound in the mammal by an imaging modality selected from the group consisting of positron emission tomography (PET) imaging, single photon emission computed tomography (SPECT), gamma camera devices, magnetic resonance imaging (MRI), and hand-held radiation detection probes.

Sodium or potassium salts of dichloroacetate (acid form dichloroacetic acid; dichloroethanoic acid) have been used to treat lactic acidosis. Also, they have been reported to be effective in inducing apoptosis, decreasing proliferation, and inhibiting tumor growth of cancer cells in vitro (Bonnet et al., Cancer Cell, January 2007;11(1):37-51). Compared to normal cells, several human cancers have higher mitochondrial membrane potential (ΔPsim) and lower expression of the K⁺ channel Kv1.5, both contributing to apoptosis resistance. Dichloroacetate (DCA) inhibits mitochondrial pyruvate dehydrogenase kinase (PDK), shifts metabolism from glycolysis to glucose oxidation, decreases ΔPsim, increases mitochondrial H₂O₂, and activates Kv channels in cancer, but not normal, cells; DCA upregulates Kv1.5 by an NFAT 1-dependent mechanism.

Any cancer characterized by having either a higher mitochondrial membrane potential (ΔPsim) than noncancerous cells of the same tissue type and species, lower expression of the mitochondrial K⁺ channel Kv1.5 than noncancerous cells of the same tissue type and species, or both can be the cancer. In one embodiment, the cancer is selected from the group consisting of non-small-cell lung cancer, glioblastoma, and breast cancer. In another embodiment, the cancer is any cancer in which dichloroacetate accumulates in cancerous cells can be the subject of the imaging method.

Any mammal can be the subject of the method. In one embodiment, the mammal is Homo sapiens. Other mammals that have economic (e.g., cow, sheep, pig) or esthetic (e.g., cat, dog, horse) utility can be the subject of the method, as well.

In addition to the compound having structure I, the composition can further contain a pharmaceutically-acceptable carrier. By “pharmaceutically-acceptable” is meant that the pharmaceutically-acceptable carrier is suitable for use in medicaments intended for administration to a mammal. Parameters which may considered to determine the pharmaceutical acceptability of a carrier can include, but are not limited to, the toxicity of the pharmaceutically-acceptable carrier, the interaction between the compound having structure I and the pharmaceutically-acceptable carrier, the approval by a regulatory body of the pharmaceutically-acceptable carrier for use in medicaments, or two or more of the foregoing, among others.

The pharmaceutically-acceptable carrier can be any material or plurality of materials which can form a composition with the compound having structure I. The particular carrier can be selected by the skilled artisan in view of the intended use of the composition and the properties of the compound having structure I, among other parameters apparent in light of the present disclosure.

Non-limiting examples of particular carriers and particular compositions follow.

In one embodiment, the pharmaceutically-acceptable carrier is water, and the composition is an aqueous solution comprising water and the compound having structure I. An example of pharmaceutically-acceptable carrier is an aqueous saline solution. The composition can further comprise solutes, such as salts, acids, bases, or mixtures thereof, among others. The composition can also comprise a surfactant, an emulsifier, or another compound capable of improving the solubility of the compound having structure I in water.

In one embodiment, the pharmaceutically-acceptable carrier is a polar organic solvent, and the composition is a polar organic solution comprising the polar organic solvent and the compound having structure I. “Polar” has its standard meaning in the chemical arts of describing a molecule that has a permanent electric dipole. A polar molecule can but need not have one or more positive, negative, or both charges. Examples of polar organic solvents include, but are not limited to, methanol, ethanol, formate, acrylate, or mixtures thereof, among others. The composition can further comprise solutes, such as salts, among others. The composition can also comprise a surfactant, an emulsifier, or another compound capable of improving the solubility of the compound having structure I in the polar organic solvent.

In one embodiment, the pharmaceutically-acceptable carrier is an apolar organic solvent, and the composition is an apolar organic solution comprising the apolar organic solvent and the compound having structure I. “Apolar” has its standard meaning in the chemical arts of describing a molecule that does not have a permanent electric dipole. The composition can further comprise solutes, such as apolar molecules, among others. The composition can also comprise a surfactant, an emulsifier, or another compound capable of improving the solubility of the compound having structure I in the apolar organic solvent. In one embodiment, the composition is a water-in-oil emulsion, wherein the compound having structure I is dissolved in water and water is emulsified into a continuous phase comprising one or more apolar organic solvents.

In one embodiment, the pharmaceutically-acceptable carrier is a solid or semisolid carrier, and the composition is a solid or semisolid matrix in or over which the compound having structure I is dispersed. Examples of components of solid carriers include, but are not limited to, sucrose, gelatin, gum arabic, lactose, methylcellulose, cellulose, starch, magnesium stearate, talc, petroleum jelly, or mixtures thereof, among others. The dispersal of the compound having structure I can be homogeneous (i.e., the distribution of the compound having structure I can be invariant across all regions of the composition) or heterogeneous (i.e., the distribution of the compound having structure I can vary at different regions of the composition). The composition can further comprise other materials, such as flavorants, preservatives, or stabilizers, among others.

In one embodiment, the pharmaceutically-acceptable carrier is a gas, and the composition can be a gaseous suspension of the compound having structure I in the gas, either at ambient pressure or non-ambient pressure. Examples of the gas include, but are not limited to, air, oxygen, nitrogen, or mixtures thereof, among others.

Other carriers will be apparent to the skilled artisan having the benefit of the present disclosure.

In addition to the compound having structure I and the pharmaceutically-acceptable carrier, and further components described above, the composition can also further comprise other compounds, such as preservatives, adjuvants, excipients, binders, other agents capable of ameliorating one or more diseases, or mixtures thereof, among others.

The concentration of the compound having structure I in the composition can vary, depending on the pharmaceutically-acceptable carrier and other parameters apparent to the skilled artisan having the benefit of the present disclosure. The concentration of other components of the composition can also vary along the same lines.

The compositions can be made up in any conventional form known in the art of pharmaceutical compounding. Exemplary forms include, but are not limited to, a solid form for oral administration such as tablets, capsules, pills, powders, granules, and the like. In one embodiment, for oral dosage, the composition is in the form of a tablet or a capsule of hard or soft gelatin, methylcellulose, or another suitable material easily dissolved in the digestive tract.

Typical preparations for intravenous administration would be sterile aqueous solutions including water/buffered solutions. Intravenous vehicles include fluid, nutrient and electrolyte replenishers. Preservatives and other additives may also be present.

In the administration step, the composition can be introduced into the mammal by any appropriate technique. An appropriate technique can vary based on the mammal, the portion of the mammal's body for which imaging is desired, and the components of the composition, among other parameters apparent to the skilled artisan having the benefit of the present disclosure. Administration can be systemic, that is, the composition is not directly delivered to a tissue, tissue type, organ, or organ system where imaging is desired, or it can be localized, that is, the composition can be directly delivered to a tissue, tissue type, organ, or organ system where imaging is desired. The route of administration can be varied, depending on the composition, the target tissue, tissue type, organ, or organ system, and other parameters, as a matter of routine experimentation by the skilled artisan having the benefit of the present disclosure. Exemplary routes of administration include transdermal, subcutaneous, intravenous, intraarterial, intramuscular, intrathecal, intraperitoneal, oral, rectal, and nasal, among others. In one embodiment, the route of administration is oral or intravenous.

By weight, suitable doses of the compound having structure I can be in the range from about 1 pg/kg body weight/day to about 10 mg/kg body weight/day. By radioactivity, suitable doses of the compound having structure I can be in the range from about 0.01 mCi/kg body weight/day to about 2.0 mCi/kg body weight/day.

The compound having structure I contains at least one radioactive tracer isotope, that is, an isotope which decays by emitting a positron, a photon, or both.

One embodiment of the present invention relates to molecular probes (compounds having structure I) analogous to dichloroacetate which target given proteins expressed by given alleles of various oncogenes, which probes can be used in an imaging modality to determine whether the given proteins are targeted by the molecular probe and hence whether dichloroacetate would be likely to be efficacious against the cancer characterized by activity of the given allele of the particular oncogene. Specifically, if the compound having structure I is observed to be concentrated in a tumor, then it is likely that dichloroacetate or another anticancer drug would be efficacious against the tumor. Imaging can also allow the clinician to observe the size, density, or other properties of the tumor, which can be useful in preparation for surgical removal of the tumor or monitoring the effect of a treatment regimen.

In another embodiment, the present invention relates to a method of treating a cancer in a mammal by administering to the mammal a composition comprising a compound having structure I, as described above.

“Treating,” as used herein in reference to cancer, is to be construed as referring to a reduction in tumor size, a reduction in cancer cell count, or a retardation of cancer progression.

The administering step can be as described above. In the event that the compound is concentrated in a tumor of the mammal suffering from the cancer, it will tend to interact with the same molecular targets as dichloroacetate and, for that reason, may have the same effects on cancer cells as dichloroacetate. Alternatively or in addition, the radioactive decay of the isotope in the compound having structure I can provide damaging doses of radiation to cancer cells.

For treating the cancer, suitable doses of the compound having structure I can be in the range from about 10 mg/kg body weight/day to about 100 mg/kg body weight/day.

In one embodiment, the cancer is selected from the group consisting of non-small-cell lung cancer, glioblastoma, and breast cancer. In another embodiment, the cancer is any cancer in which dichloroacetate accumulates can be the subject of the imaging method.

In one embodiment, the present invention relates to a method of imaging in a mammal a medical condition by administering to the mammal a composition comprising a compound having structure I, as described above, and observing the distribution of the compound in the mammal by an imaging modality selected from the group consisting of positron emission tomography (PET) imaging, single photon emission computed tomography (SPECT), gamma camera devices, magnetic resonance imaging (MRI), and hand-held radiation detection probes.

The composition and the administering and observing steps can be as described above. The medical condition can be a disease characterized by activity of pyruvate dehydrogenase kinase. In one embodiment, the medical condition is lactic acidosis.

In another embodiment, the medical condition is the function of an organ, whereby imaging allows observations of the organ's function, whether normal, diseased, or otherwise impaired. In one embodiment, the medical condition is selected from the group consisting of cardiac function and cardiovascular function.

It should be appreciated by those of skill in the art that the techniques disclosed herein represent techniques discovered by the inventors to function well in the practice of the invention. However, those of skill in the art should, in light of the present disclosure, appreciate that many changes can be made in the specific embodiments which are disclosed and still obtain a like or similar result without departing from the spirit and scope of the invention.

What is claimed is:

1. A compound, having structure I: 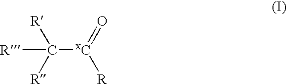
wherein:
^{x}C is selected from the group consisting of ¹¹C, ¹²C, and ¹³C;
R is selected from the group consisting of —OH, —O⁻R⁴⁺ wherein R⁴ is selected from the group consisting of Na and K, —OR³ wherein R³ is selected from the group consisting of C₁₋₈ alkyl and C₁₋₈ alkenyl, —NH₂, and NHR³;
R′ is selected from the group consisting of —H, —Cl, —F, —Br, and —I;
R″ is selected from the group consisting of —H, —Cl, —F, —Br, and —I; and
R′″ is selected from the group consisting of —H, —Cl, —F, —Br, —I, ¹⁸F, ¹⁹F, ⁷⁵Br, ⁷⁶Br, ⁷⁷Br, ¹²³I, ¹²⁴I, and ¹³¹I;
provided, when ^{x}C is ¹²C, R′″ is selected from the group consisting of ¹⁸F, ¹⁹F, ⁷⁵Br, ⁷⁶Br, ⁷⁷ Br, ¹²³I, ¹²⁴I and ¹³¹I.

2. The compound of claim 1, selected from the group consisting of (i) the compound having structure I, wherein ^{x}C is ¹¹C, R is —O⁻R⁴⁺ wherein R⁴ is selected from the group consisting of Na and K, R′ is —Cl, R″ is —H, and R′″ is —Cl; (ii) the compound having structure I, wherein ^{x}C is ¹²C, R is —O⁻R⁴⁺ wherein R⁴ is selected from the group consisting of Na and K, R′ is —Cl, R″ is —H, and R′″ is ¹⁸F; (iii) the compound having structure I, wherein ^{x}C is ¹²C, R is —O⁻R⁴⁺ wherein R⁴ is selected from the group consisting of Na and K, R′ is —Cl, R″ is —Cl, and R′″ is ¹⁸F; (iv) the compound having structure I, wherein ^{x}C is ¹²C, R is —O⁻R⁴⁺ wherein R⁴ is selected from the group consisting of Na and K, R′ is —Cl, R″ is —H, and R′″ is ⁷⁵Br; (v) the compound having structure I, wherein ^{x}C is ¹²C, R is —O⁻R⁴⁺ wherein R⁴ is selected from the group consisting of Na and K, R′ is —Cl, R″ is —H, and R′″ is ⁷⁶Br; (vi) the compound having structure I, wherein ^{x}C is ¹²C, R is —O⁻R⁴⁺ wherein R⁴ is selected from the group consisting of Na and K, R′ is —Cl, R″ is —H, and R′″ is ⁷⁷Br; (vii) the compound having structure I, wherein ^{x}C is ¹²C, R is —O⁻R⁴⁺ wherein R⁴ is selected from the group consisting of Na and K, R′ is —Cl, R″ is —Cl, and R′″ is ⁷⁵Br; (viii) the compound having structure I, wherein ^{x}C is ¹²C, R is —O⁻R⁴⁺ wherein R⁴ is selected from the group consisting of Na and K, R′ is —Cl, R″ is —Cl, and R′″ is ⁷⁶Br; (ix) the compound having structure I, wherein ^{x}C is ¹²C, R is —O⁻R⁴⁺ wherein R⁴ is selected from the group consisting of Na and K, R′ is —Cl, R″ is —Cl, and R′″ is ⁷⁷Br; (x) the compound having structure I, wherein ^{x}C is ¹²C, R is —O⁻R⁴⁺ wherein R⁴ is selected from the group consisting of Na and K, R′ is —Cl, R″ is —H, and R′″ is ¹²³I; (xi) the compound having structure I, wherein ^{x}C is ¹²C, R is —O⁻R⁴⁺ wherein R⁴ is selected from the group consisting of Na and K, R′ is —Cl, R″ is —H, and R′″ is ¹²⁴I; (xii) the compound having structure I, wherein ^{x}C is ¹²C, R is —O⁻R⁴⁺ wherein R⁴ is selected from the group consisting of Na and K, R′ is —Cl, R″ is —H, and R′″ is ¹³¹I; (xiii) the compound having structure I, wherein ^{x}C is ¹²C, R is —O⁻R⁴⁺ wherein R⁴ is selected from the group consisting of Na and K, R′ is —Cl, R″ is —Cl, and R′″ is ¹²³I; (xiv) the compound having structure I, wherein ^{x}C is ¹²C, R is —O⁻R⁴⁺ wherein R⁴ is selected from the group consisting of Na and K, R′ is —Cl, R″ is —Cl, and R′″ is ¹²⁴I; and (xv) the compound having structure I, wherein ^{x}C is ¹²C, R is —O⁻R⁴⁺ wherein R⁴ is selected from the group consisting of Na and K, R′ is —Cl, R″ is —Cl, and R′″ is ¹³¹I.

3. A method of imaging in a mammal a cancer, comprising:
administering to the mammal a composition comprising a compound having structure I: 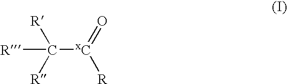
wherein ^{x}C is selected from the group consisting of ¹¹C, ¹²C, and ¹³C; R is selected from the group consisting of —OH, —O⁻R⁴⁺ wherein R⁴ is selected from the group consisting of Na and K, —OR³ wherein R³ is selected from the group consisting of C₁₋₈ alkyl and C₁₋₈ alkenyl, —NH₂, and NHR³; R′ is selected from the group consisting of —H, —Cl, —F, —Br, and —I; R″ is selected from the group consisting of —H, —Cl, —F, —Br, and —I; and R′″ is selected from the group consisting of —H, —Cl, —F, —Br, —I, ¹⁸F, ¹⁹F, ⁷⁵Br, ⁷⁶Br, ⁷⁷Br, ¹²³I, ¹²⁴I, and ¹³¹I; provided, when ^{x}C is ¹²C, R′″ is selected from the group consisting of ¹⁸F, ¹⁹F, ⁷⁵Br, ⁷⁶Br, ⁷⁷Br, ¹²³I, ¹²⁴I, and ¹³¹I; and
observing the distribution of the compound in the mammal by an imaging modality selected from the group consisting of positron emission tomography (PET) imaging, single photon emission computed tomography (SPECT), gamma camera devices, magnetic resonance imaging (MRI), and hand-held radiation detection probes.

4. The method of claim 3, wherein the compound is selected from the group consisting of (i) the compound having structure I, wherein ^{x}C is ¹¹C, R is —O⁻R⁴⁺ wherein R⁴ is selected from the group consisting of Na and K, R′ is —Cl, R″ is —H, and R′″ is —Cl; (ii) the compound having structure I, wherein ^{x}C is ¹²C, R is —O⁻R⁴⁺ wherein R⁴ is selected from the group consisting of Na and K, R′ is —Cl, R″ is —H, and R′″ is ¹⁸F; (iii) the compound having structure I, wherein ^{x}C is ¹²C, R is —O⁻R⁴⁺ wherein R⁴ is selected from the group consisting of Na and K, R′ is —Cl, R″ is —Cl, and R′″ is ¹⁸F; (iv) the compound having structure I, wherein ^{x}C is ¹²C, R is —O⁻R⁴⁺ wherein R⁴ is selected from the group consisting of Na and K, R′ is —Cl, R″ is —H, and R′″ is ⁷⁵Br; (v) the compound having structure I, wherein ^{x}C is ¹²C, R is —O⁻R⁴⁺ wherein R⁴ is selected from the group consisting of Na and K, R′ is —Cl, R″ is —H, and R′″ is ⁷⁶Br; (vi) the compound having structure I, wherein ^{x}C is ¹²C, R is —O⁻R⁴⁺ wherein R⁴ is selected from the group consisting of Na and K, R′ is —Cl, R″ is —H, and R′″ is ⁷⁷Br; (vii) the compound having structure I, wherein ^{x}C is ¹²C, R is —O⁻R⁴⁺ wherein R⁴ is selected from the group consisting of Na and K, R′ is —Cl, R″ is —Cl, and R′″ is ⁷⁵Br; (viii) the compound having structure I, wherein ^{x}C is ¹²C, R is —O⁻R⁴⁺ wherein R⁴ is selected from the group consisting of Na and K, R′ is —Cl, R″ is —Cl, and R′″ is ⁷⁶Br; (ix) the compound having structure I, wherein ^{x}C is ¹²C, R is —O⁻R⁴⁺ wherein R⁴ is selected from the group consisting of Na and K, R′ is —Cl, R″ is —Cl, and R′″ is ⁷⁷Br; (x) the compound having structure I, wherein ^{x}C is ¹²C, R is —O⁻R⁴⁺ wherein R⁴ is selected from the group consisting of Na and K, R′ is —Cl, R″ is —H, and R′″ is ¹²³I; (xi) the compound having structure I, wherein ^{x}C is ¹²C, R is —O⁻R⁴⁺ wherein R⁴ is selected from the group consisting of Na and K, R′ is —Cl, R″ is —H, and R′″ is ¹²⁴I; (xii) the compound having structure I, wherein ^{x}C is ¹²C, R is —O⁻R⁴⁺ wherein R⁴ is selected from the group consisting of Na and K, R′ is —Cl, R″ is —H, and R′″ is ¹³¹I; (xiii) the compound having structure I, wherein ^{x}C is ¹²C, R is —O⁻R⁴⁺ wherein R⁴ is selected from the group consisting of Na and K, R′ is —Cl, R″ is —Cl, and R′″ is ¹²³I; (xiv) the compound having structure I, wherein ^{x}C is ¹²C, R is —O⁻R⁴⁺ wherein R⁴ is selected from the group consisting of Na and K, R′ is —Cl, R″ is —Cl, and R′″ is ¹²⁴I; and (xv) the compound having structure I, wherein ^{x}C is ¹²C, R is —O⁻R⁴⁺ wherein R⁴ is selected from the group consisting of Na and K, R′ is —Cl, R″ is —Cl, and R′″ is ¹³¹I.

5. The method of claim 3, wherein the cancer is one in which dichloroacetate accumulates in the cancerous cells.

6. The method of claim 3, wherein the cancer is selected from the group consisting of non-small-cell lung cancer, glioblastoma, and breast cancer.

7. A method of treating a cancer in a mammal, comprising:
administering to the mammal a composition comprising a compound having structure I: 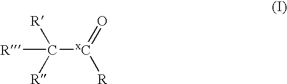
wherein ^{x}C is selected from the group consisting of ¹¹C, ¹²C, and ¹³C; R is selected from the group consisting of —OH, —O⁻R⁴⁺ wherein R⁴ is selected from the group consisting of Na and K, —OR³ wherein R³ is selected from the group consisting of C₁₋₈ alkyl and C₁₋₈alkenyl, —NH₂, and NHR³; R′ is selected from the group consisting of —H, —Cl, —F, —Br, and —I; R″ is selected from the group consisting of —H, —Cl, —F, —Br, and —I; and R′″ is selected from the group consisting of —H, —Cl, —F, —Br, —I, ¹⁸F, ¹⁹F, ⁷⁵Br, ⁷⁶Br, ⁷⁷Br, ¹²³I, ¹²⁴I, and ¹³¹I; provided, when ^{x}C is ¹²C, R′″ is selected from the group consisting of ¹⁸F, ¹⁹F, ⁷⁵Br, ⁷⁶Br, ⁷⁷Br, ¹²³I, ¹²⁴I, and ¹³¹I.

8. The method of claim 7, wherein the compound is selected from the group consisting of (i) the compound having structure I, wherein ^{x}C is ¹¹C, R is —O⁻R⁴⁺ wherein R⁴ is selected from the group consisting of Na and K, R′ is —Cl, R″ is —H, and R′″ is —Cl; (ii) the compound having structure I, wherein ^{x}C is ¹²C, R is —O⁻R⁴⁺ wherein R⁴ is selected from the group consisting of Na and K, R′ is —Cl, R″ is —H, and R′″ is ¹⁸F; (iii) the compound having structure I, wherein ^{x}C is ¹²C, R is —O⁻R⁴⁺ wherein R⁴ is selected from the group consisting of Na and K, R′ is —Cl, R″ is —Cl, and R′″ is ¹⁸F; (iv) the compound having structure I, wherein ^{x}C is ¹²C, R is —O⁻R⁴⁺ wherein R⁴ is selected from the group consisting of Na and K, R′ is —Cl, R″ is —H, and R′″ is ⁷⁵Br; (v) the compound having structure I, wherein ^{x}C is ¹²C, R is —O⁻R⁴⁺ wherein R⁴ is selected from the group consisting of Na and K, R′ is —Cl, R″ is —H, and R′″ is ⁷⁶Br; (vi) the compound having structure I, wherein ^{x}C is ¹²C, R is —O⁻R⁴⁺ wherein R⁴ is selected from the group consisting of Na and K, R′ is —Cl, R″ is —H, and R′″ is ⁷⁷Br; (vii) the compound having structure I, wherein ^{x}C is ¹²C, R is —O⁻R⁴⁺ wherein R⁴ is selected from the group consisting of Na and K, R′ is —Cl, R″ is —Cl, and R′″ is ⁷⁵Br; (viii) the compound having structure I, wherein ^{x}C is ¹²C, R is —O⁻R⁴⁺ wherein R⁴ is selected from the group consisting of Na and K, R′ is —Cl, R″ is —Cl, and R′″ is ⁷⁶Br; (ix) the compound having structure I, wherein ^{x}C is ¹²C, R is —O⁻R⁴⁺ wherein R⁴ is selected from the group consisting of Na and K, R′ is —Cl, R″ is —Cl, and R′″ is ⁷⁷Br; (x) the compound having structure I, wherein ^{x}C is ¹²C, R is —O⁻R⁴⁺ wherein R⁴ is selected from the group consisting of Na and K, R′ is —Cl, R″ is —H, and R′″ is ¹²³I; (xi) the compound having structure I, wherein ^{x}C is ¹²C, R is —O⁻R⁴⁺ wherein R⁴ is selected from the group consisting of Na and K, R′ is —Cl, R″ is —H, and R′″ is ¹²⁴I; (xii) the compound having structure I, wherein ^{x}C is ¹²C, R is —O⁻R⁴⁺ wherein R⁴ is selected from the group consisting of Na and K, R′ is —Cl, R″ is —H, and R′″ is ¹³¹I; (xiii) the compound having structure I, wherein ^{x}C is ¹²C, R is —O⁻R⁴⁺ wherein R⁴ is selected from the group consisting of Na and K, R′ is —Cl, R″ is —Cl, and R′″ is ¹²³I; (xiv) the compound having structure I, wherein ^{x}C is ¹²C, R is —O⁻R⁴⁺ wherein R⁴ is selected from the group consisting of Na and K, R′ is —Cl, R″ is —Cl, and R′″ is ¹²⁴I; and (xv) the compound having structure I, wherein ^{x}C is ¹²C, R is —O^{—}R⁴⁺ wherein R⁴ is selected from the group consisting of Na and K, R′ is —Cl, R″ is —Cl, and R′″ is ¹³¹I.

9. The method of claim 7, wherein the cancer is one in which dichloroacetate accumulates in the cancerous cells.

10. The method of claim 7, wherein the cancer is selected from the group consisting of non-small-cell lung cancer, glioblastoma, and breast cancer.

11. A method of imaging in a mammal a medical condition, comprising:
administering to the mammal a composition comprising a compound having structure I: 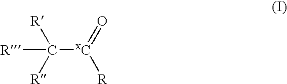
wherein ^{x}C is selected from the group consisting of ¹¹C, ¹²C, and ¹³C; R is selected from the group consisting of —OH, —O⁻R⁴⁺ wherein R⁴ is selected from the group consisting of Na and K, —OR³ wherein R³ is selected from the group consisting of C₁₋₈ alkyl and C₁₋₈ alkenyl, —NH₂, and NHR³; R′ is selected from the group consisting of —H, —Cl, —F, —Br, and —I; R″ is selected from the group consisting of —H, —Cl, —F, —Br, and —I; and R′″ is selected from the group consisting of —H, —Cl, —F, —Br, —I, ¹⁸F, ¹⁹F, ⁷⁵Br, ⁷⁶Br, ⁷⁷Br, ¹²³I, ¹²⁴I, and ¹³¹I; provided, when ^{x}C is ¹²C, R′″ is selected from the group consisting of ¹⁸F, ¹⁹F, ⁷⁵Br, ⁷⁶Br, ⁷⁷Br, ¹²³I, ¹²⁴I, and ¹³¹I; and
observing the distribution of the compound in the mammal by an imaging modality selected from the group consisting of positron emission tomography (PET) imaging, single photon emission computed tomography (SPECT), gamma camera devices, magnetic resonance imaging (MRI), and hand-held radiation detection probes.

12. The method of claim 11, wherein the compound is selected from the group consisting of (i) the compound having structure I, wherein ^{x}C is ¹¹C, R is —O⁻R⁴⁺ wherein R⁴ is selected from the group consisting of Na and K, R′ is —Cl, R″ is —H, and R′″ is —Cl; (ii) the compound having structure I, wherein ^{x}C is ¹²C, R is —O⁻R⁴⁺ wherein R⁴ is selected from the group consisting of Na and K, R′ is —Cl, R″ is —H, and R′″ is ¹⁸F; (iii) the compound having structure I, wherein ^{x}C is ¹²C, R is —O⁻R⁴⁺ wherein R⁴ is selected from the group consisting of Na and K, R′ is —Cl, R″ is —Cl, and R′″ is ¹⁸F; (iv) the compound having structure I, wherein ^{x}C is ¹²C, R is —O⁻R⁴⁺ wherein R⁴ is selected from the group consisting of Na and K, R′ is —Cl, R″ is —H, and R′″ is ⁷⁵Br; (v) the compound having structure I, wherein ^{x}C is ¹²C, R is —O⁻R⁴⁺ wherein R⁴ is selected from the group consisting of Na and K, R′ is —Cl, R″ is —H, and R′″ is ⁷⁶Br; (vi) the compound having structure I, wherein ^{x}C is ¹²C, R is —O⁻R⁴⁺ wherein R⁴ is selected from the group consisting of Na and K, R′ is —Cl, R″ is —H, and R′″ is ⁷⁷Br; (vii) the compound having structure I, wherein ^{x}C is ¹²C, R is —O⁻R⁴⁺ wherein R⁴ is selected from the group consisting of Na and K, R′ is —Cl, R″ is —Cl, and R′″ is ⁷⁵Br; (viii) the compound having structure I, wherein ^{x}C is ¹²C, R is —O⁻R⁴⁺ wherein R⁴ is selected from the group consisting of Na and K, R′ is —Cl, R″ is —Cl, and R′″ is ⁷⁶Br; (ix) the compound having structure I, wherein ^{x}C is ¹²C, R is —O⁻R⁴⁺ wherein R⁴ is selected from the group consisting of Na and K, R′ is —Cl, R″ is —Cl, and R′″ is ⁷⁷Br; (x) the compound having structure I, wherein ^{x}C is ¹²C, R is —O⁻R⁴⁺ wherein R⁴ is selected from the group consisting of Na and K, R′ is —Cl, R″ is —H, and R′″ is ¹²³I; (xi) the compound having structure I, wherein ^{x}C is ¹²C, R is —O⁻R⁴⁺ wherein R⁴ is selected from the group consisting of Na and K, R′ is —Cl, R″ is —H, and R′″ is ¹²⁴I; (xii) the compound having structure I, wherein ^{x}C is ¹²C, R is —O⁻R⁴⁺ wherein R⁴ is selected from the group consisting of Na and K, R′ is —Cl, R″ is —H, and R′″ is ¹³¹I; (xiii) the compound having structure I, wherein ^{x}C is ¹²C, R is —O⁻R⁴⁺ wherein R⁴ is selected from the group consisting of Na and K, R′ is —Cl, R″ is —Cl, and R′″ is ¹²³I; (xiv) the compound having structure I, wherein ^{x}C is ¹²C, R is —O⁻R⁴⁺ wherein R⁴ is selected from the group consisting of Na and K, R′ is —Cl, R″ is —Cl, and R′″ is ¹²⁴I; and (xv) the compound having structure I, wherein ^{x}C is ¹²C, R is —O⁻R⁴⁺ wherein R⁴ is selected from the group consisting of Na and K, R′ is —Cl, R″ is —Cl, and R′″ is ¹³¹I.

13. The method of claim 11, wherein the medical condition is lactic acidosis.

14. The method of claim 11, wherein the medical condition is selected from the group consisting of cardiac function and cardiovascular function.
